(19) United States
(12) Patent Application Publication
(10) Pub. No.: US 2018/0340222 A1
(43) Pub. Date: Nov. 29, 2018

(54) SOL-GEL DERIVED BIOHYBRID MATERIALS INCORPORATING LONG-CHAIN DNA APTAMERS

(71) Applicant: McMaster University, Hamilton (CA)

(72) Inventors: Carmen Carrasquilla, Hamilton (CA); Yingfu Li, Dundas (CA); John D. Brennan, Dundas (CA)

(73) Assignee: McMaster University, Hamilton (CA)

(21) Appl. No.: 15/989,894

(22) Filed: May 25, 2018

Related U.S. Application Data
(60) Provisional application No. 62/511,466, filed on May 26, 2017.

(51) Int. Cl. C12Q 1/6876; B01J 13/00

(52) U.S. Cl. CPC C12Q 1/6876; B01J 13/0008

(57) ABSTRACT

The present application relates to a new class of macroporous bio/inorganic hybrids, engineered through a high-throughput materials screening approach that entrap micron-sized concatemeric DNA aptamers. The entrapment of these long-chain DNA aptamers allows their retention within the macropores of the silica material, so that aptamers can interact with high molecular weight targets such as proteins.

### RELATED APPLICATIONS

This application claims priority to U.S. Provisional Patent Application No. 62/511,466 filed on May 26, 2017, the content of which is hereby incorporated by reference in its entirety.

### INCORPORATION OF SEQUENCE LISTIING

A computer readable form of the Sequence Listing “3244-P53314US01_SequenceListing.txt” (4,096 bytes), submitted via EFS-WEB and created on May 24, 2018, is herein incorporated by reference.

### FIELD

The present application relates to bio/inorganic hybrid materials produced by entrapment of micron-sized concatemeric nucleic acid molecules in macroporous sol-gels.

### BACKGROUND

The sol-gel process has been widely used to entrap biomolecules into porous materials to produce various analytical devices.^{[1]} In all cases, the retention of entrapped biomolecules is based on size exclusion and required materials with mesopore diameters of under 10 nm. As such, these bio/inorganic hybrid materials are restricted to interactions with molecules less than 2 kDa,[^{2]} as larger targets are unable to access the entrapped biomolecules.^{[3]}

To extend this approach to larger analytes, it is necessary to produce materials with macroporous morphologies. To prevent leaching of biomolecules, it is possible to immobilize them to the surface of the material using covalent or affinity-based interactions.^{[4]} However, these methods require multiple time-consuming steps, can lead to biomolecule denaturation, and have lower loading capacity compared to sol-gel entrapment.

### SUMMARY

The present inventors have demonstrated an alternative method for entrapping biomolecular species that are large enough to remain immobilized even in micron-sized pores. Rolling circle amplification (RCA) is a biochemical reaction that can produce very large single-stranded DNA amplicons that contain a repetitive DNA sequence.^{[5]} When the circular DNA template is designed to contain the complementary sequence of a DNA aptamer, its RCA reaction will generate long strands of DNA containing tandem repeats of an aptamer sequence,[^{6]} which are referred to as concatemeric DNA aptamers herein. The present inventors have demonstrated that megadalton-sized assemblies of concatemeric aptamers can be entrapped into specially-designed macroporous sol-gel derived organosilicate composites with high target-binding activity and minimal leaching, allowing for fabrication of flow-through biosensors for targets ranging from small molecules to proteins.

Accordingly, the present disclosure provides a bio/inorganic hybrid material comprising: a) a macroporous sol-gel; and b) one or more concatemeric nucleic acid molecules entrapped within a) having a molecular weight greater than 10,000 Da and comprising tandem repeating functional nucleic acid sequences. In an embodiment, the macroporous sol-gel is a macroporous sol-gel derived silicate, organosilicate composite or other metal oxide or mixed metal oxide composite. In an embodiment, the concatemeric nucleic acid molecules comprise sequences for one or more RNA aptamers or DNA aptamers or DNAzymes or aptazymes, or a combination thereof.

Also provided is a biosensor comprising a) the bio/inorganic hybrid material disclosed herein, further comprising b) nucleic acid molecules complementary to at least one portion of the concatemeric nucleic acid molecule labelled with a detectable label for detection of an analyte. The detectable label for detection of the analyte may be suitable for a fluorescent system, a colorimetric system, Raman, infrared or other optical system, and an electrochemical system. In one embodiment, the detectable label for detection of the analyte is a fluorescent label.

In a particular embodiment, b) comprises fluorophore labelled nucleic acid molecules complementary to a portion of the concatemeric nucleic acid molecules and b) further comprises nucleic acid molecules complementary to a second portion of the concatemeric nucleic acid molecules conjugated to a quencher; wherein the quencher quenches the fluorophore in the absence of the analyte. In this embodiment, the fluorophore labelled nucleic acid and the quencher conjugated nucleic acid hybridize to the portions of the concatemeric nucleic acid molecule to form a quenched concatemer/DNA duplex in the absence of analyte and in the presence of analyte, the duplex undergoes a conformational change resulting in the quencher conjugated nucleic acid to be released and the analyte binding to a portion of the concatemeric nucleic acid molecule resulting in a fluorescence signal due to the fluorophore labelled nucleic acid molecules remaining hybridized to the portion of the concatemeric nucleic acid molecules.

The present disclosure also provides a method for preparing a bio/inorganic hybrid material, comprising a) combining concatemeric nucleic acid molecules with a sol-gel precursor; and b) incubating a) under conditions to form a macroporous metal oxide or organically-modified metal oxide gel. In an embodiment, b) comprises forming the macroporous metal oxide or organically-modified metal oxide gel into bulk monoliths, monolithic capillary columns, thin films, or arrays.

In an embodiment, the sol-gel precursor comprises sodium silicate (SS), tetramethoxysilane (TMOS) or TMOS and methyltrimethoxysilane (MTMS) in a 60:40 volume percent ratio with 1.25 to 5% weight-to-volume of 600 to 8000 Da poly(ethylene glycol). In one embodiment, the sol-gel precursor comprises a mixture of tetramethoxysilane and methyltrimethoxysilane in a 60:40 volume percent ratio with 5 percent weight-to-volume 6,000 Da poly(ethylene glycol).

Also provided herein is a method of preparing a biosensor comprising a) combining concatemeric nucleic acid molecules and nucleic acid molecules complementary to a portion of the concatemeric nucleic acid molecule labelled with a detectable label for detection of an analyte, and b) mixing a) with a sol-gel precursor under conditions to form a macroporous metal oxide or organically-modified metal oxide gel.

In an embodiment, the method further comprises in a) combining nucleic acid molecules complementary to a second portion of the concatemeric nucleic acid molecule conjugated to a quencher; wherein the quencher quenches the fluorophore in the absence of the analyte. In this embodiment, the nucleic acid molecules complementary to a portion of the concatemeric nucleic acid molecule labelled with a detectable label for detection of an analyte remain hybridized with the concatemeric nucleic acid molecules in the presence of the analyte.

Further provided herein is a monolithic capillary column comprising the bio/inorganic hybrid material disclosed herein or the biosensor disclosed herein, within a hollow fused silica capillary.

The present disclosure also provides assay methods that utilize the bio/inorganic hybrid material, biosensor or the monolithic capillary column disclosed herein. In one embodiment, the disclosure provides a method of detecting one or more analytes in a sample, comprising:

a) mixing the sample with the biosensor material disclosed herein; and

b) detecting the labelled nucleic acid molecules; wherein detecting the labelled nucleic acid molecules indicates the presence of the one or more analytes.

In an embodiment, in a) the analyte binds to the concatemeric molecules, displacing the quencher conjugated nucleic acid molecules, allowing the fluorophore-labelled nucleic acid molecules to be detected in b). In this embodiment, the analyte binding causes a conformational change that releases the quencher conjugated nucleic acid molecule.

In another embodiment, the disclosure provides a method of detecting one or more analytes in a sample, comprising:

a) flowing the sample through a monolithic column comprising the biosensor material disclosed herein; and

b) monitoring for detection, wherein a positive result indicates the presence of the one or more analytes in the sample.

In an embodiment, detection in b) comprises a fluorescent system and the positive result is a presence of a fluorescent signal. In a particular embodiment, the monolithic column comprises the biosensor material comprising the labelled nucleic acid molecules complementary to a portion of the concatemeric nucleic acid molecule, and in a) the analyte binds to the concatemeric nucleic acid molecules displacing the labelled nucleic acid molecules complementary to a portion of the concatemeric nucleic acid molecule; and b) comprises collecting eluate from the column and detecting the fluorescence of the eluate.

In an embodiment, the analyte is selected from metal ions, small molecules, drugs, hormonal growth factors, biomolecules, toxins, peptides, proteins, viruses, bacteria, and cells.

In yet another embodiment, the disclosure provides a method of separating one or more target molecules from a sample, comprising:

a) capturing the one or more target molecules using a monolithic column comprising the bio/inorganic hybrid material disclosed herein by flowing the sample through said monolithic column, wherein the bio/inorganic hybrid material comprises concatemeric nucleic acid molecules that are able to bind to the target molecule; and

b) optionally isolating the one or more molecules from the monolithic column. In an embodiment, the concatemeric nucleic acid molecules that are able to bind to the target molecule comprise aptamers.

The target molecule may be selected from metal ions, small molecules, drugs, hormonal growth factors, biomolecules, toxins, peptides, proteins, viruses, bacteria, and cells.

Even further provided are kits comprising the bio/inorganic material, the biosensor and the monolithic capillary column disclosed herein.

Other features and advantages of the present disclosure will become apparent from the following detailed description. It should be understood, however, that the detailed description and the specific examples, while indicating embodiments of the application, are given by way of illustration only and the scope of the claims should not be limited by these embodiments, but should be given the broadest interpretation consistent with the description as a whole.

### BRIEF DESCRIPTION OF THE DRAWINGS

Embodiments are described below in relation to the drawings in which:

FIG. 1 shows opacity plots of sol-gel derived materials. Percent transmittance of a) SS, b) TMOS, c) 40% MTMS (in TMOS), and d) MTMS sols mixed in assay buffer with 0-10 kDa PEG at various concentrations after 3 h gelation at room temperature. Materials with transmittance 20% are considered to be macroporous.

FIG. 2 shows aptamer leaching from sol-gel derived materials. The percent leaching of a) ATP concatemers versus monomers and b) PDGF concatemers versus monomers entrapped in various mesoporous or macroporous sol-gel derived materials. c) Schematic of concatemers entrapped within a macroporous matrix.

FIG. 3 shows the signal response comparison of aptamer reporters. Fluorescence from a) ATP-binding monomer and concatemer with 2 mM ATP or b) PDGF-binding monomer and concatemer with 200 nM PDGF in various mesoporous or macroporous materials. The black line in a) and b) indicates a normalized F/Fo of 1.0 (no signal increase). Response of c) ATP-binding concatemer with 0-3 mM ATP or d) PDGF-binding concatemer with 0-300 nM PDGF in Meso or Macro 40% MTMS.

FIG. 4 shows SEM images of sol-gel derived monolithic columns. Magnified images of monolithic columns with or without entrapped concatemeric aptamers formed in a 250 μm i.d. capillary using SEM analysis of non-conductive materials.

FIG. 5 shows target detection using aptameric monolithic columns. a) Schematic of macroporous sol-gel derived monolith containing concatemeric aptamers and target binding-induced release of F′DNA. b) ATP concatemer column response to 0-3 mM ATP or c) PDGF concatemer column response to 0-300 nM PDGF. Insets: representative fluorescence scans of eluate fractions upon target addition—b) 2 mM ATP or c) 200 nM PDGF (RFU=relative fluorescence units).

FIG. 6 shows time-resolved changes in transmittance of sol-gel derived materials. Transmittance at 400 nm of SS (top) and 40% MTMS (bottom) mixed in assay buffer with 0-10 kDa PEG at various concentrations over 12 has samples undergo phase separation and evolve over time.

FIG. 7 shows DLS measurements of DNA constructs in solution. Hydrodynamic size distributions of a) concatemeric, b) monomeric and c) circular template constructs for the ATP aptamer (top) and PDGF aptamer (bottom) in solution as measured by dynamic light scattering intensity.

FIG. 8 shows signal enhancement of concatemeric reporters in PEG-doped buffer. Fluorescence signal response of the a) concatemeric ATP aptamer with 2 mM ATP and b) concatemeric PDGF aptamer with 200 nM PDGF, in solution containing increasing concentrations of 1-10 kDa PEG.

FIG. 9 shows the selectivity of entrapped concatemeric aptamers. Selectivity of the a) concatemeric ATP aptamer to different nucleotides at 2 mM concentration and b) concatemeric PDGF aptamer to different growth factors and proteins at 200 nM concentration, entrapped in Macro 40% MTMS.

FIG. 10 shows concatemeric aptamer emission intensity upon exposure to DNase I. Fluorescence measurements over time (1 h) after addition of 1 unit of DNase I to the ATP-binding and PDGF-binding concatemeric aptamer reporters in solution or entrapped within the Macro 40% MTMS material.

FIG. 11 shows backpressure of monolithic columns upon aging. Backpressure of monolithic columns relative to empty capillaries (indicated by a relative backpressure of 1 at ˜15 PSI) after various aging periods at a flow rate of 1 μL/min. Columns were used for target detection assays only after at least 7 days of aging.

FIG. 12 shows EDX analysis of sol-gel derived monolithic columns. A spectral overlay comparing the differences in elemental composition between a monolithic column with versus without entrapped concatemeric aptamers. Inset: the relative atomic contribution from C, N, O and Si for a sol-gel derived monolithic column with or without entrapped concatemeric aptamer amplicons.

FIG. 13 shows fluorescence scans of column fractions. Emission scans of eluate from monolithic columns containing various entrapped DNA molecules, divided into four fractions: a) pre-wash 1, b) pre-wash 2, c) elution with target and d) post-target wash. Fractions 1, 2 and 4 serve as wash steps with buffer only and fractions 3 contains either 2 mM ATP (top) or 200 nM PDGF (bottom) for the appropriate column.

FIG. 14 shows the column response to PDGF in a complex sample. PDGF concatemer-doped monolithic column response to 0 or 200 nM PDGF in either buffer or undiluted human serum.

FIG. 15 shows the response of mixed concatemer columns. Columns containing both PDGF-FAM and ATP-Cy5 concatemers were exposed to either 2 mM ATP (black line), 200 nM PDGF (grey dashed line) or a mixture of 200 nM PDGF and 2 mM ATP in buffer (grey line). Left spectra show emission of FAM originating from the PDGF aptamer, right spectra show emission of Cy5 originating from the ATP aptamer.

### DETAILED DESCRIPTION

Unless otherwise indicated, the definitions and embodiments described in this and other sections are intended to be applicable to all embodiments and aspects of the present disclosure herein described for which they are suitable as would be understood by a person skilled in the art.

In understanding the scope of the present disclosure, the term “comprising” and its derivatives, as used herein, are intended to be open ended terms that specify the presence of the stated features, elements, components, groups, integers, and/or steps, but do not exclude the presence of other unstated features, elements, components, groups, integers and/or steps. The foregoing also applies to words having similar meanings such as the terms, “including”, “having” and their derivatives. The term “consisting” and its derivatives, as used herein, are intended to be closed terms that specify the presence of the stated features, elements, components, groups, integers, and/or steps, but exclude the presence of other unstated features, elements, components, groups, integers and/or steps. The term “consisting essentially of”, as used herein, is intended to specify the presence of the stated features, elements, components, groups, integers, and/or steps as well as those that do not materially affect the basic and novel characteristic(s) of features, elements, components, groups, integers, and/or steps.

As used herein, the singular forms “a”, “an” and “the” include plural references unless the content clearly dictates otherwise. The modifier “about” used in connection with a quantity is inclusive of the stated value and has the meaning dictated by the context (e.g., it includes the degree of error associated with measurement of the particular quantity). When referring to a period such as a year or annually, it includes a range from 9 months to 15 months. All ranges disclosed herein are inclusive of the endpoints, and the endpoints are independently combinable with each other.

### Bio/Inorganic Hybrid Materials, Biosensors, Columns and Methods of Making Same

The present disclosure provides a bio/inorganic hybrid material comprising: a) a macroporous sol-gel and b) one or more concatemeric nucleic acid molecules entrapped within a) having a molecular weight greater than 10,000 Da and comprising tandem repeating functional nucleic acid sequences.

The term “sol-gel” as used herein refers to any material prepared using a sol-gel process. The sol-gel process is a wet-chemical technique used for the fabrication of both glassy and ceramic materials. In this process, the sol (or solution) evolves gradually towards the formation of a gel-like network containing both a liquid phase and a solid phase. Precursors are, for example, metal alkoxides or metal chlorides, which undergo hydrolysis and polycondensation reactions to form a colloid. The basic structure or morphology of the solid phase can range anywhere from discrete colloidal particles to continuous chain-like polymer networks, depending on the identity of the precursors and any additives. In an embodiment, the precursors are silicon or titanium alkoxides or chlorides.

In an embodiment of the application, the sol-gel is prepared from a sodium silicate precursor solution. The preparation of sodium silicate solutions for use as a sol-gel precursor is known in the art.¹⁴ In still further embodiments, the sol-gel is prepared from organic polyol silane precursors. Examples of the preparation of sol-gels from organic polyol silane precursors are described in “Polyol-Modified Silanes as Precursors for Silica”, U.S. patent application publication no. US2004/0034203 filed on Jun. 2, 2003, the contents of which are incorporated herein by reference. In further embodiments the sol-gel precursors are reacted in the presence of additives that control the morphology and size of the resulting sol-gel, such as those described in “Methods and Compounds for Controlling the Morphology and Shrinkage of Silica Derived from Polyol-Modified Silanes”, U.S. CIP patent application publication no. US2004/0249082 filed on Apr. 1, 2004, the contents of which are incorporated herein by reference. In some embodiments, tetraalkoxysilane is reacted with a methylsilsesquioxane (MSQ) precursor as described in U.S. Pat. No. 7,582,214, incorporated herein by reference in its entirety. Briefly, the MSQ precursor may be any compound that may be hydrolyzed, then condensed to form MSQ materials. Such compounds will have the general formula Me-Si—(OR)₃, wherein R is a group that may be hydrolyzed under acidic or basic conditions to provide free OH groups that may be polycondensed to form MSQ materials. In an embodiment of the invention, R is methyl or ethyl, suitably methyl.

In specific embodiments of the application, the sol-gel precursors are reacted in the presence of a polymer additive to promote phase separation by spinodal decomposition prior to gelation.

In an embodiment, the polymer additive is selected from any such compound and includes, but is not limited to, for example, polyethylene glycol (PEG); amino-terminated polyethylene glycol (PEG-NH₂);

polypropylene glycol (PPG), polypropylene glycol bis(2-amino-propyl ether) (PPG-NH₂); polyalcohols, for example, polyvinyl alcohol; polysaccharides; poly(vinyl pyridine); polyacids, for example, poly(acrylic acid); polyacrylamides e.g. poly(N-isopropylacrylamide) (polyNIPAM), or polyallylamine (PAM), or mixtures thereof. In embodiment of the application the polymer additive is PEG. In still further embodiments, the polymer additive is PEG, for example PEG having a molecular weight between about 500-100000 Da, suitably between about 500 and 20000 Da, more suitably between about 600 and 10000 Da.

In an embodiment, the macroporous sol-gel is a macroporous sol-gel derived silicate, organosilicate composite or other metal oxide or mixed metal oxide composite.

Macroporosity may be assessed by measuring the transmittance of a material at 400 nm, with materials with transmittance below 20% being macroporous. As shown in the examples, many materials comprised of sodium silicate (SS), TMOS (tetramethoxysilane) or 40 vol % methyltrimethoxysilane (MTMS) in TMOS with variable amounts of 600 to 8000 Da poly(ethylene glycol) PEG, such as 1.25 to 5% weight-to-volume, demonstrated such transmittance values. In one embodiment, the sol-gel precursor comprises a mixture of tetramethoxysilane and methyltrimethoxysilane in a 60:40 volume percent ratio with 5 percent weight-to-volume 6,000 Da poly(ethylene glycol).

As used herein, the term “immobilized” of “entrapped” or synonyms thereof, means that movement of the referenced component of the biosensor, is restricted.

The term “nucleic acid” refers to polynucleotides such as deoxyribonucleic acid (DNA) and ribonucleic acid (RNA).

The term “rolling circle amplification” or “RCA” as used herein refers to a unidirectional nucleic acid replication that can rapidly synthesize multiple copies of circular molecules of DNA or RNA. In an embodiment, rolling circle amplification is an isothermal enzymatic process where a short DNA or RNA primer is amplified to form a long single stranded DNA or RNA using a circular DNA template and an appropriate DNA or RNA polymerase.

The term “concatemeric nucleic acid molecule” as used herein refers to a long polynucleotide that is the product of the RCA process and contains tandem repeating sequences that are complementary to the circular template. The tandem repeating functional nucleic acid sequences are optionally aptamers or aptazymes, which may be formed by rolling circle amplification or RCA.

Accordingly, in an embodiment, the concatemeric nucleic acid molecules comprise sequences for one or more RNA aptamers or DNA aptamers or DNAzymes or aptazymes, or a combination thereof.

The term “aptamer” as used herein refers to short, chemically synthesized, single stranded (ss) RNA or DNA oligonucleotides which fold into specific three-dimensional (3D) structures that bind to a specific analyte with dissociation constants, for example, in the pico- to nano-molar range.

Also provided is a biosensor comprising a) the bio/inorganic hybrid material disclosed herein, further comprising b) nucleic acid molecules complementary to at least one portion of the concatemeric nucleic acid molecule labelled with a detectable label for detection of an analyte. In one embodiment, the portion of the concatemeric nucleic acid molecules that the nucleic acid molecules are complementary to comprises a region that is susceptible to a conformational change that releases the nucleic acid molecules of b) upon binding of an analyte.

The term “analyte” as used herein means any agent for which one would like to sense or detect using a biosensor of the present application. The term analyte also includes mixtures of compounds or agents such as, but not limited to, combinatorial libraries and samples from an organism or a natural environment. In an embodiment, the analyte is selected from metal ions, small molecules, drugs, hormonal growth factors, biomolecules, toxins, peptides, proteins, viruses, bacteria, and cells.

The detectable label for detection of the analyte may be suitable for a fluorescent system, a colorimetric system, Raman, infrared or other optical system, and an electrochemical system. In one embodiment, the detectable label for detection of the analyte is a fluorescent label, such as FAM or Cy5.

In a particular embodiment, b) comprises fluorophore labelled nucleic acid molecules complementary to a portion of the concatemeric nucleic acid molecules and b) further comprises nucleic acid molecules complementary to a second portion of the concatemeric nucleic acid molecules conjugated to a quencher; wherein the quencher quenches the fluorophore in the absence of the analyte. In this embodiment, the fluorophore labelled nucleic acid and the quencher conjugated nucleic acid hybridize to the portions of the concatemeric nucleic acid molecule to form a quenched concatemer/DNA duplex in the absence of analyte and in the presence of analyte, the duplex undergoes a conformational change resulting in the quencher conjugated nucleic acid to be released and the analyte binding to a portion of the concatemeric nucleic acid molecule resulting in a fluorescence signal due to the fluorophore labelled nucleic acid molecules remaining hybridized to the portion of the concatemeric nucleic acid molecules.

The present disclosure also provides a method for preparing a bio/inorganic hybrid material, comprising a) combining concatemeric nucleic acid molecules with a sol-gel precursor disclosed herein; and b) incubating a) under conditions to form a macroporous metal oxide or organically-modified metal oxide gel. In an embodiment, b) comprises forming the macroporous metal oxide or organically-modified metal oxide gel into bulk monoliths, monolithic capillary columns, thin films, or arrays.

Also provided herein is a method of preparing a biosensor comprising a) combining concatemeric nucleic acid molecules and nucleic acid molecules complementary to a portion of the concatemeric nucleic acid molecule labelled with a detectable label for detection of an analyte; and b) mixing a) with a sol-gel precursor under conditions to form a macroporous metal oxide or organically-modified metal oxide gel.

By “under conditions to form a macroporous metal oxide or organically-modified metal oxide gel” it is meant the conditions used herein to effect hydrolysis and condensation of the sol-gel precursors. This includes, in aqueous solution, at a pH in the range of 4-11.5, specifically in the range 5-10, and temperatures in the range of 0-80° C., and specifically in the range 0-40° C., and optionally with sonication and/or in the presence of catalysts known to those skilled in the art, including acids, amines, dialkyltin esters, titanates, etc. As disclosed herein, the precursors are combined with an additive which causes spinodal decomposition (phase transition) before gelation, to provide macroporous silica matrixes, optionally PEG.

In an embodiment, the method further comprises in a) combining nucleic acid molecules complementary to a second portion of the concatemeric nucleic acid molecule conjugated to a quencher; wherein the quencher quenches the fluorophore in the absence of the analyte. In this embodiment, the nucleic acid molecules complementary to a portion of the concatemeric nucleic acid molecule labelled with a detectable label for detection of an analyte remain hybridized with the concatemeric nucleic acid molecule in the presence of the analyte.

Further provided herein is a monolithic capillary column comprising the bio/inorganic hybrid material disclosed herein or the biosensor disclosed herein, within a hollow fused silica capillary.

The term “monolithic capillary column” as used herein refers to a cylindrical stationary phase comprising a self-supporting monolith or continuous chain-like polymer network of sol-gel derivatized within a hollow fused silica capillary. In order to prepare the column, the sol-gel “solution” (the precursors and concatemeric nucleic acids/nucleic acids) is placed in a capillary prior to gelation and allowed to gel within the walls of the capillary.

The term “hollow fused silica capillary” as used herein refers to a circular cross-section tube having an inner wall and an outer wall and inner diameters ranging from 10 μm to 1000 μm. The tube wall may be made of fused silica, metal, plastic and other materials. When the tube wall is made of fused silica, the wall of the capillary possesses terminal Si—OH groups which can undergo a condensation reaction with terminal Si—OH or Si—OR groups on the silica-based monolithic capillary column within to produce a covalent “Si—O—Si” linkage between the monolith and the capillary wall. This provides a column with structural integrity that maintains the monolith within the column.

### Assays

The present disclosure also provides assay methods that utilize the bio/inorganic hybrid material, biosensor or the monolithic capillary column disclosed herein. In one embodiment, the disclosure provides a method of detecting one or more analytes in a sample, comprising:

a) mixing the sample with the biosensor material disclosed herein; and

b) detecting the labelled nucleic acid molecules; wherein detecting the labelled nucleic acid molecules indicates the presence of the one or more analytes.

In an embodiment, in a) the analyte binds to the concatemeric molecules, displacing the quencher conjugated nucleic acid molecules, allowing the fluorophore-labelled nucleic acid molecules to be detected in b). In this embodiment, the fluorophore labelled nucleic acid and the quencher conjugated nucleic acid hybridize to the portions of the concatemeric nucleic acid molecule to form a quenched concatemer/DNA duplex in the absence of analyte and in the presence of analyte, the duplex undergoes a conformational change resulting in the quencher conjugated nucleic acid to be released and the analyte binding to the portion of the concatemeric nucleic acid molecule resulting in a fluorescence signal due to the fluorophore labelled nucleic acid molecules remaining hybridized to the portion of the concatemeric nucleic acid molecules.

In another embodiment, the disclosure provides a method of detecting one or more analytes in a sample, comprising:

a) flowing the sample through a monolithic column comprising the biosensor material disclosed herein; and

b) monitoring for detection, wherein a positive result indicates the presence of the one or more analytes in the sample.

In an embodiment, detection in b) comprises a fluorescent system and the positive result is a presence of a fluorescent signal. In a particular embodiment, the monolithic column comprises the biosensor material comprising the labelled nucleic acid molecules complementary to a portion of the concatemeric nucleic acid molecule, and in a) the analyte binds to the concatemeric nucleic acid molecules displacing the labelled nucleic acid molecules complementary to a portion of the concatemeric nucleic acid molecule; and b) comprises collecting eluate from the column and detecting the fluorescence of the eluate.

In an embodiment, the analyte is selected from metal ions, small molecules, drugs, hormonal growth factors, biomolecules, toxins, peptides, proteins, viruses, bacteria, and cells.

The term “sample(s)” as used herein refers to any material that one wishes to assay using the biosensor of the application. The sample may be from any source, for example, any biological (for example human or animal medical samples), environmental (for example water or soil) or natural (for example plants) source, or from any manufactured or synthetic source (for example food or drinks). The sample is one that comprises or is suspected of comprising one or more analytes.

In an embodiment, the sample further comprises a nuclease inhibitor.

In yet another embodiment, the disclosure provides a method of separating one or more target molecules from a sample, comprising:

a) capturing the one or more target molecules using a monolithic column comprising the bio/inorganic hybrid material disclosed herein by flowing the sample through said monolithic column, wherein the bio/inorganic hybrid material comprises concatemeric nucleic acid molecules that are able to bind to the target molecule; and

b) optionally isolating the one or more molecules from the monolithic column. In an embodiment, the concatemeric nucleic acid molecules that are able to bind to the target molecule comprise aptamers.

The target molecule may be selected from metal ions, small molecules, drugs, hormonal growth factors, biomolecules, toxins, peptides, proteins, viruses, bacteria, and cells.

### Kits

Even further provided are kits comprising the bio/inorganic material, biosensor and the monolithic capillary column disclosed herein.

In some embodiments, the kit includes instructions for using the material, biosensor or column in the assay and any controls needed to perform the assay. The controls may be on the biosensor itself, or alternatively, on a separate substrate. The kit may further comprise wash solutions, eluent, and other reagents that may be required for the assay.

The above disclosure generally describes the present application. A more complete understanding can be obtained by reference to the following specific examples. These examples are described solely for the purpose of illustration and are not intended to limit the scope of the application. Changes in form and substitution of equivalents are contemplated as circumstances might suggest or render expedient. Although specific terms have been employed herein, such terms are intended in a descriptive sense and not for purposes of limitation.

The following non-limiting examples are illustrative of the present disclosure:

### EXAMPLES

The present inventors set out to identify appropriate porous material for entrapping concatemeric aptamers. The morphology of sol-gel materials is affected by several parameters that control gelation time and phase separation, which include types of silica precursors and polymer additives, reaction pH and ionic strength.^{[3a,3c,7]} The present inventors made adaptation to a previously reported screening approach^{[7]} to identify suitable compositions that could retain concatemeric aptamers with minimal leaching, allow concatemer accessibility to both small molecule and protein targets, produce self-supporting monolithic capillary columns, and allow pressure-driven flow through a capillary column with low backpressure.

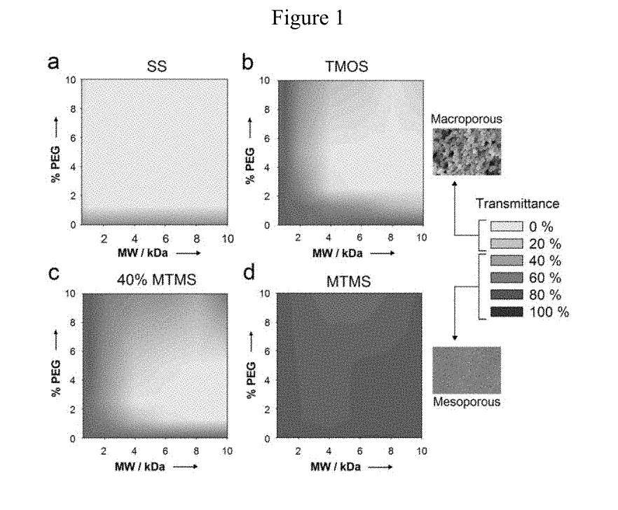

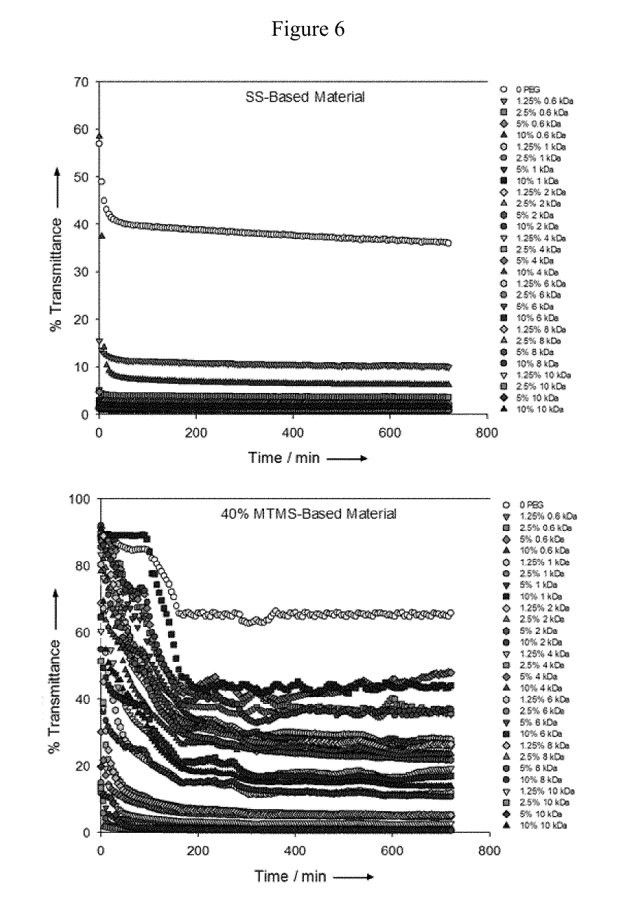

A total of 140 formulations were prepared from four silica precursors—sodium silicate (SS), tetramethoxysilane (TMOS), methyltrimethoxysilane (MTMS) or 40 vol % MTMS in TMOS, which were previously used for entrapping functional aptamers.^{[8]} Each precursor was combined with five concentrations (0, 1.25, 2.5, 5, 10% w/v) of seven poly(ethylene glycol) (PEG) species varying in MW. Macroporosity was assessed by measuring the transmittance of each material at 400 nm, which decreases owing to increased light scattering as materials become more macroporous (FIG. 6). A cutoff of 20% transmittance was selected, below which materials were considered to be macroporous.^{[7]} FIG. 1 demonstrates that transmittance (and hence morphology) can be carefully controlled by adjusting the precursor and polymer additive properties. Many materials comprised of SS, TMOS or 40 vol % MTMS in TMOS with variable amounts of PEG demonstrated transmittance values indicative of macroporosity and formed self-supporting monoliths without flocculation, and thus were further investigated.

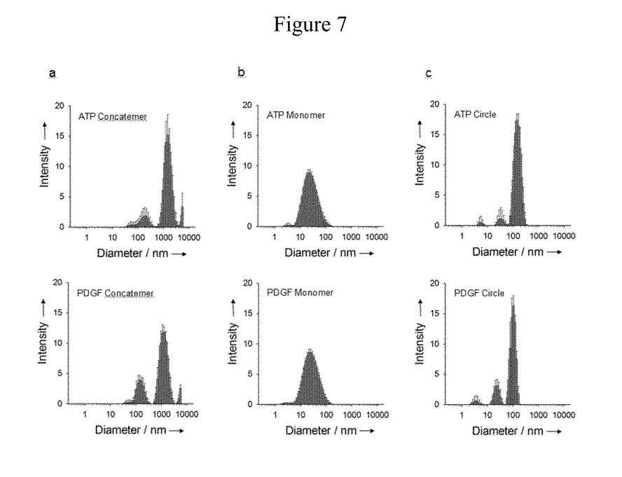

To monitor the effects of entrapment on the performance of aptamers (e.g. leaching, target-binding ability), two structure-switching DNA aptamers were chosen, one for adenosine triphosphate (ATP), and another for the platelet-derived growth factor (PDGF) protein^{[9]} (see Table 1 for the DNA sequences used).^{[10]} In this design, fluorophore and quencher-labelled DNA strands (FDNA and QDNA, respectively) hybridize to the monomeric or concatemeric aptamers to form a quenched aptamer/DNA duplex. Upon binding its target, this duplex undergoes a conformational change to release the QDNA and produce a fluorescence signal enhancement. Dynamic light scattering measurements of monomeric and concatemeric aptamers in solution (FIG. 7) demonstrated that the average hydrodynamic diameter of the concatemeric aptamers was ˜1.5 μm, which was much higher than the size of monomeric aptamers (˜20 nm if fully extended). The small peak at ˜100 nm comes from the circular DNA template.

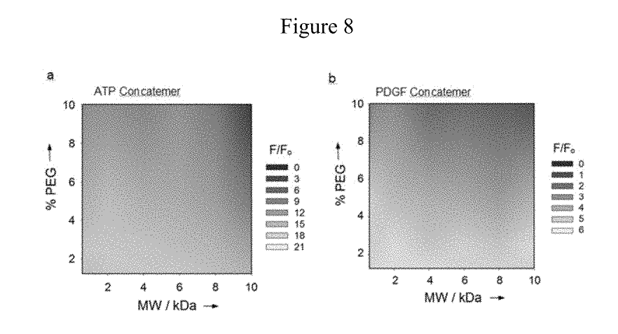

The effect of PEG on the structure-switching ability of aptamers in solution was examined, as it was reported that high levels of PEG can prevent hybridization of FDNA and QDNA with the aptamers.^{[11]} FIG. 8 demonstrates that reduced signal enhancement occurs with increasing concentration and MW of PEG. Therefore, a subset of 24 macroporous materials, with low to intermediate MWs and concentrations of PEG, were chosen for aptamer entrapment (Table 2).

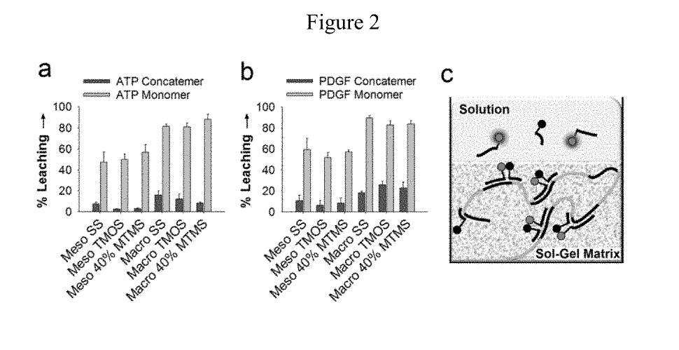

FIG. 2 shows the leaching of entrapped monomeric and concatemeric aptamers, as determined by the fluorescence intensity of the supernatant used to wash the monoliths. Monomeric aptamers demonstrated substantial leaching from mesoporous materials (50%) and almost complete leaching from macroporous materials (90%), showing that leaching increases with pore size. Conversely, concatemeric aptamers demonstrated significantly lower leaching from all materials (20% or less) due to their larger size relative to monomers, and fluorescence polarization studies revealed that the intensity arose from dehybridized FDNA rather than loss of concatemeric aptamers (Table 3), indicating efficient entrapment of concatemeric aptamers in macroporous materials. Based on these results, further studies focused on a subset of three macroporous materials: SS with 5% of 0.6 kDa PEG (Macro SS), TMOS with 5% 6 kDa PEG (Macro TMOS), and 40% MTMS with 5% 6 kDa PEG (Macro 40% MTMS). Corresponding mesoporous materials were also tested, which had identical precursor compositions but lacked PEG.

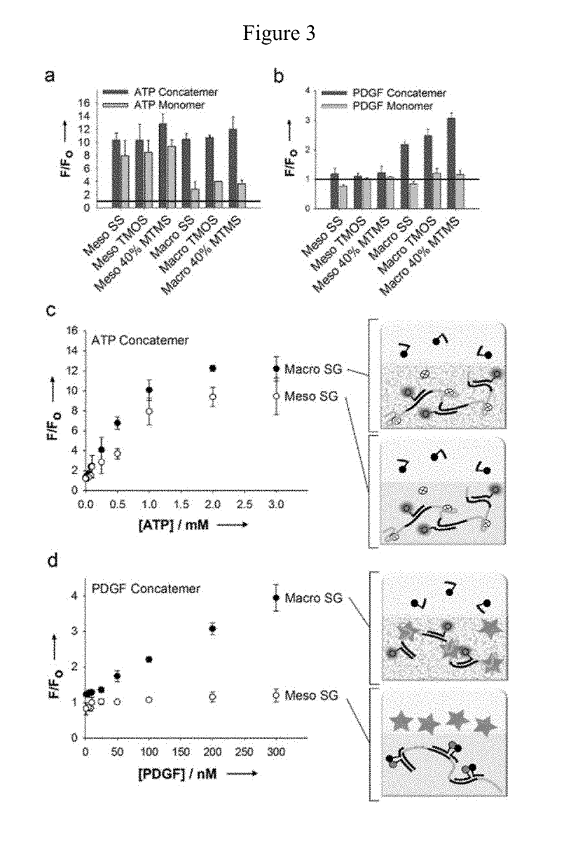

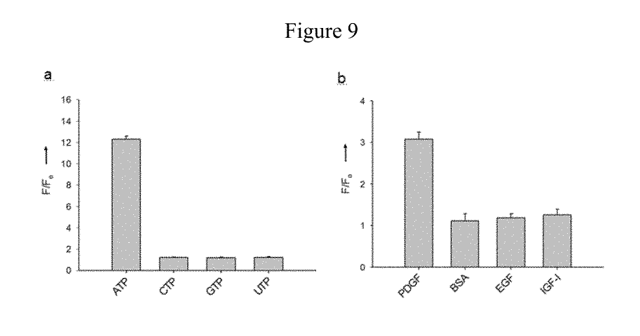

Signal response of entrapped monomeric and concatemeric aptamers were next evaluated when exposed to their cognate targets (2 mM ATP or 200 nM PDGF), which would depend on both access of the analyte to the entrapped aptamer and the capacity of the aptamer to retain structure-switching ability. In mesoporous materials, both monomeric and concatemeric versions of the ATP aptamer showed a similar response to ATP (˜8-10-fold increase in signal, FIG. 3a) while addition of PDGF to entrapped monomeric and concatemeric PDGF aptamers produced no increase in fluorescence (FIG. 3b), indicating that the PDGF was unable to enter the mesoporous material, as expected. However, macroporous materials containing concatemeric aptamers demonstrated substantial fluorescence enhancements, with an 8-fold enhancement for the ATP aptamer (FIG. 3a) and up to a 3-fold enhancement for the PDGF aptamer (FIG. 3b). In stark contrast, addition of cognate targets to macroporous materials containing monomeric aptamers produced much lower fluorescence enhancements, consistent with loss of the entrapped aptamers via leaching, while addition of unintended targets to entrapped concatemeric aptamers produced no signal (FIG. 9), demonstrating retention of the expected selectivity. These results conclusively demonstrate that entrapment of long-chain DNA aptamers in optimized macroporous materials allows detection of targets spanning small molecules to proteins.

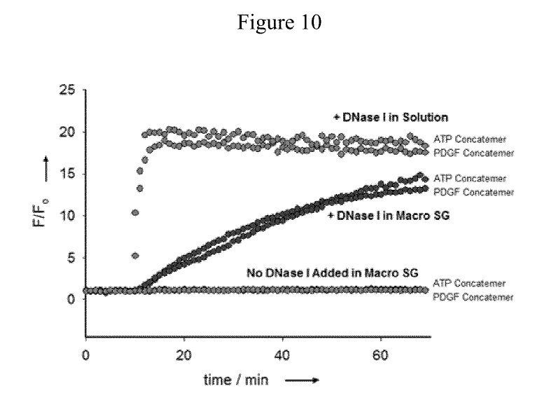

Concentration-dependent signal responses of concatemeric ATP (FIG. 3c) and PDGF (FIG. 3d) aptamers entrapped in Meso and Macro 40% MTMS were also examined. While ATP could induce a concentration-dependent fluorescence enhancement in both materials, PDGF was only able to cause a signal change in the macroporous material, further confirming that large targets such as PDGF require macropores to access the entrapped aptamers. Interestingly, while aptamers remained accessible to proteins, their entrapment within macropores did afford some protection against degradation by nucleases (>60 min for full degradation when entrapped vs. ˜1 min in solution, FIG. 10). Inclusion of nuclease inhibitors into samples should further reduce nuclease degradation.

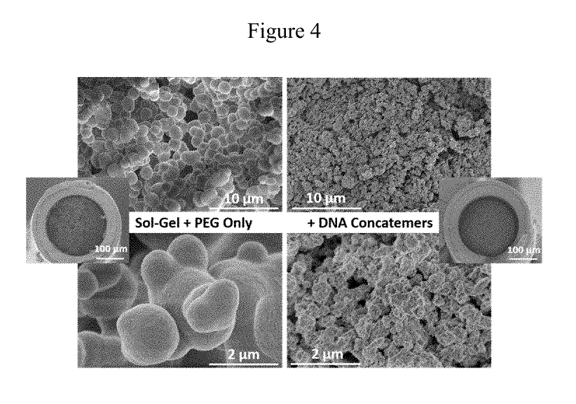

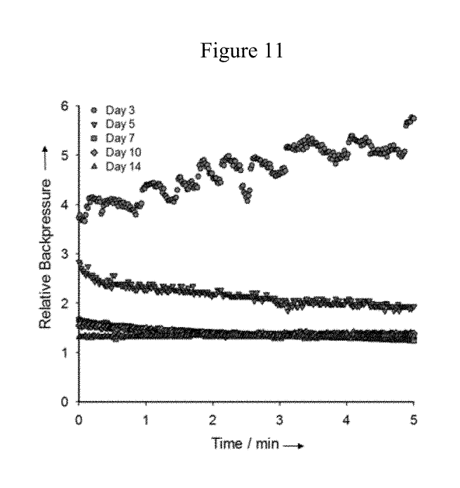

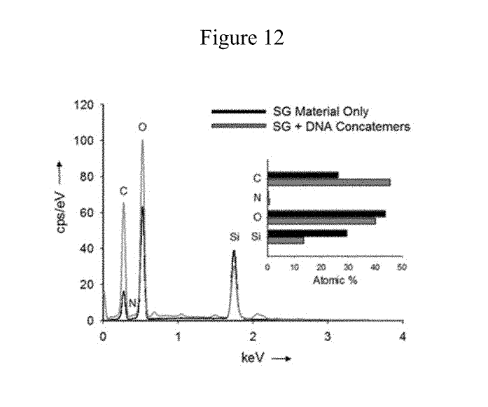

As a practical demonstration of the utility of the macroporous materials, aptamer-doped monolithic columns were produced within fused silica capillaries using the 40% MTMS material for use as flow-through biosensors. The columns could withstand flow rates up to 30 μL/min, though at very high backpressures, but were typically utilized at a flow rate of 1 μL/min, which allowed operation at a low backpressure (FIG. 11). Scanning electron microscopy (SEM) was used to image the structure of monolithic columns with and without entrapped concatemeric aptamers (FIG. 4). Undoped columns showed macropores on the order of 1-2 μm in diameter. Columns with concatemeric aptamers showed a substantially different structure, with the appearance of roughly spherical DNA structures coating the silica particles. Energy dispersive x-ray spectroscopy (EDX) was used to compare the elemental composition of columns with or without entrapped concatemers (FIG. 12). In columns containing concatemeric aptamers, the decreased contribution from silicon and increased carbon and nitrogen content support the conclusion that the nanostructures observed using SEM are in fact long-chain DNA aptamers coating the silica skeleton.

Taken together, the SEM and EDX data show that the aptamers are likely adsorbed or partially entrapped in the materials, with a significant amount of the concatemer exposed to the pore solvent. Previous fluorescence studies of entrapped aptamers^{[12]} show that aptamers remain highly mobile when entrapped, indicating that the anionic DNA is not strongly adsorbed to either silica or organosilicate surfaces, and thus retains structure-switching ability. Thus, without wishing to be bound by theory, the aptamers are likely retained primarily as a result of size exclusion, where large concatemers are simply too big to elute even through micron scale pores.

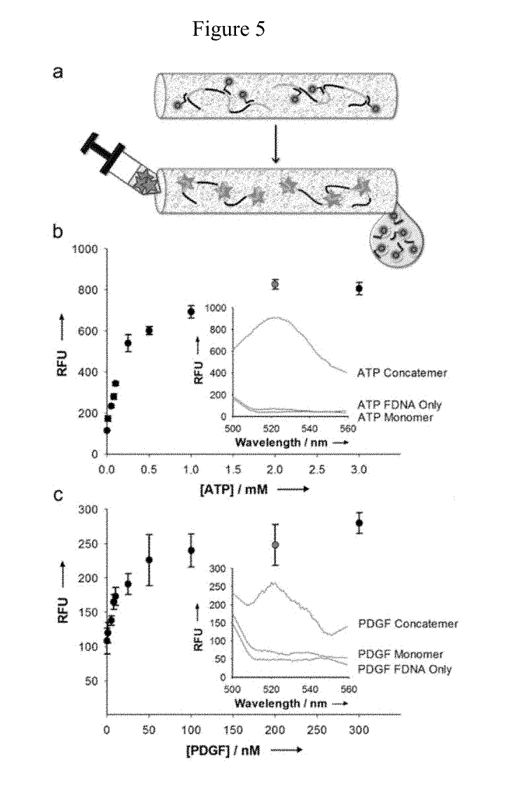

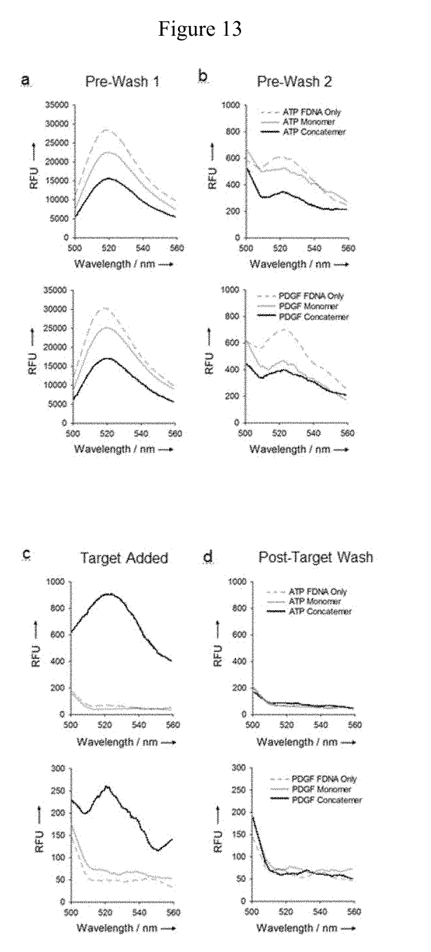

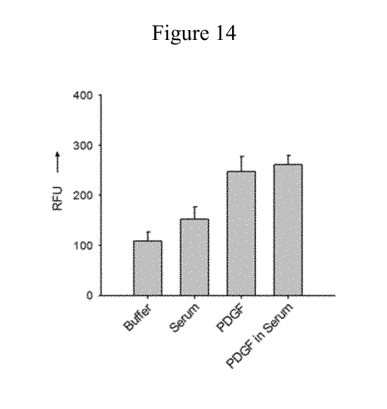

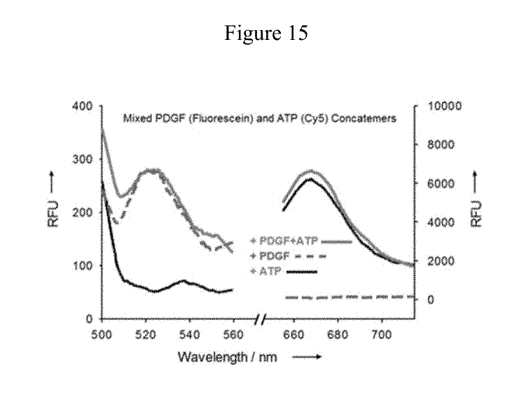

To achieve flow-through sensing, the quencher in the QDNA strand was replaced with a fluorophore (FAM or Cy5) to produce F′DNA or F″DNA, respectively, and the original FDNA was not included. In this configuration, the F′DNA (or F″DNA) is released upon target binding and produces a fluorescence spike upon elution (FIG. 5a). Upon addition of cognate target, the column eluate showed a concentration-dependent fluorescence increase (FIG. 13) for both ATP (FIG. 5b) and PDGF (FIG. 5c) systems, while addition of targets to columns with entrapped monomers or F′DNA alone resulted in no fluorescence (FIGS. 5b and 5c, insets). PDGF could also be detected when present in blood serum (FIG. 14) and the columns could be used to entrap both concatemeric aptamers simultaneously (PDGF-FAM and ATP-Cy5) for multiplexed detection (FIG. 15). Hence, the entrapment of concatemeric aptamers into macroporous columns enables fabrication of flow-based sensors for a range of targets, and may also be amenable to affinity based purification or evaluation of aptamer-target binding constants using well known chromatographic methods.[^{13]}

### Materials and Methods

Chemicals. Standard and functionalized DNA oligonucleotides were synthesized and purified by HPLC by Integrated DNA Technologies (Coralville, Iowa). Adenosine 5′-triphosphate (ATP), cytidine 5′-triphosphate (CTP), guanosine 5′-triphosphate (GTP), uridine 5′-triphosphate (UTP), deoxyribonuclease I (DNase I), T4 polynucleotide kinase (PNK; with 10× reaction buffer A), T4 DNA ligase (with 10× T4 DNA ligase buffer), 10 mM dNTPs, phi29 DNA polymerase (with 10× phi29 DNA polymerase buffer), GeneRuler™ 1 kb Plus DNA ladder and 10,000× SYBR Safe DNA gel stain were purchased from Fermentas Life Sciences (Burlington, ON). Recombinant human platelet derived growth factor (PDGF), epidermal growth factor (EGF) and insulin-like growth factor I (IGF-I) were purchased from Cedarlane (Burlington, ON). Sodium silicate solution (SS solution, ultrapure grade, ˜14% Na₂O, ˜29% silica) was purchased from Fisher Scientific (Pittsburgh, Pa.). Human serum (sterile-filtered from male AB clotted whole blood), bovine serum albumin (BSA), poly(ethylene) glycol (PEG, 600-10,000 Da), tetramethoxysilane (TMOS), methyltrimethoxysilane (MTMS), and Dowex 50×8-100 cation exchange resin and all other analytical grade chemicals and solvents were purchased from Sigma-Aldrich (Oakville, ON). Water was purified prior to use with a Millipore Milli-Q Synthesis A10 water purification system.

Preparation of Concatemeric DNA Aptamers and Reporter Complexes. Concatemer constructs of each structure-switching aptamer and the fluorescence-signaling aptamer reporter complexes for ATP and PDGF binding were prepared using the sequences given in Table 1 as described elsewhere^{[6]} and briefly below. The linear circular templates were first phosphorylated using 10 U of T4 PNK and 100 nmol ATP at 37° C. for 30 min followed by heating at 90° C. for 5 min and cooling to room temperature. These were then ligated using 15 U of T4 DNA ligase at room temperature for 12 h in 1× ligase buffer. The circularized templates were ethanol precipitated before purifying on a 10% polyacrylamide gel, ethanol precipitated and resuspended in water.

The RCA reaction of each aptamer sequence was carried out by heating 10 pmol of circular template with 10 pmol of primer and 5 μL of 10× phi29 polymerase buffer (36.5 μL total volume) at 90° C. for 1 min. Following cooling, 2.5 μL of 10 μM dNTPs and 10 U of phi29 polymerase were added to the reaction mixture and allowed to incubate at 30° C. for 1 h. The reaction was followed by a 5-fold dilution with water before heating at 90° C. for 5 min to deactivate the enzyme. The concatemeric aptamers from the reaction mixture were purified by centrifugation using a 100 kDa Nanosep® spin column and quantified using a NanoVue spectrophotometer (absorbance at 260 nm). As the exact size of the concatemer construct is unknown, its approximate molar concentration was obtained based on one repeat of the monomeric aptamer sequence.

Tripartite reporter complexes were prepared by combining either the concatemeric or monomeric aptamer with its FDNA and QDNA in a 1:1:6 molar ratio (100 nM final concentration of FDNA), respectively, in assay buffer (40 mM Tris.HCl, 200 mM NaCl, 4 mM MgCl₂ at pH 7.8). Previous work determined that using a 1:1 aptamer/FDNA ratio with 6× QDNA produced the greatest amount of quenching for low initial background fluorescence prior to target binding and a sensitivity and selectivity similar to the monomeric aptamer reporter systems.¹ These solutions were then heated at 90° C. for 5 minutes, cooled and incubated for 30 minutes at room temperature.

Optimization of Concatemer Fluorescence Signaling in Solution. To study the effects of PEG on signaling in solution, the reporter system solutions were prepared at 2× concentration then mixed in a 1:1 ratio with 1.25-10% PEG (w/v, final) of 1-10 kDa. Baseline fluorescence was measured for 10 min prior to the addition of target. Target analyte for each aptamer was then added at a final concentration of 2 mM ATP or 200 nM PDGF to the appropriate system and fluorescence measurements were continued.

Preparation of Sols and Monolithic Silica Disks. The silica and organosilicate precursors SS, TMOS and MTMS, were used to prepare the sols for aptamer entrapment studies as described elsewhere.^{[8]} Sodium silicate sols were prepared by diluting 2.59 g of a stock SS solution to 10 mL with water, mixing the solution with 5.5 g DOWEX for 2 min to bring the pH to ˜4, and then vacuum filtering this solution through a Buchner funnel to remove the resin followed by further filtration through a 0.2 μm membrane syringe filter to remove any particulates in the solution. Before use, 120 g of the Dowex resin was cleaned by stirring in 150 mL 0.1 N HCl for 1 h, followed by vacuum filtration and washing with water until the filtrate ran clear to ensure that the final pH of the sol solution was close to 4.0 (in order to form consistent final materials). To make TMOS and MTMS sols, 700 μL of water and 50 μL HCl (0.1 N) were added to 2.25 mL TMOS or MTMS and then sonicated for 20 min in ice-cold water. The 60% TMOS—40% MTMS (v/v) mixture were prepared by proportionally dividing the 2.25 mL of silane to 1350 μL TMOS and 900 μL MTMS, mixing with water and acid and co-hydrolyzing in an ultrasonic bath, as described above. All prepared sols were stored on ice until use and used within 1 h.

Monoliths for opacity screening were prepared by combining each of the sols with 2× PEG-doped assay buffer in a 1:1 (v/v) ratio, depositing 50 μL of the mixtures in a 96-well plate and allowing it to gel for 3 h prior to absorbance measurements at 400 nm using a Tecan M1000 multimode plate reader. Poly(ethylene glycol) with various molecular weights ranging from 0.6-10 kDa, was used at five final concentrations from 0-10% (w/v). Kinetic analysis of transmittance was done for the SS and 60% TMOS—40% MTMS (v/v) mixture sols with 0-10% (w/v) of 0.6-10 kDa PEG in a 96 well-plate by measuring absorbance at 400 nm every 5 min for 12 h.

Entrapment of Concatemeric Aptamer Complexes in Sol-Gel Derived Disks. Tripartite aptamer complexes (in a 1:1:6 Aptamer/FDNA/QDNA molar ratio) for entrapment were prepared at 2× final concentration in 2× PEG-doped assay buffer, heated at 90° C. for 5 min, cooled at room temperature and mixed in a 1:1 volume ratio with a freshly-prepared sol at room temperature. The aptamer-sol mixtures were deposited into the wells of a 96-well plate at a volume of 50 μL per well and allowed to gel and age for at least 3 h and then overlaid with assay buffer prior to washing and analysis.

The various sol-gel derived materials containing the reporter complexes were washed three times with 50 μL buffer at room temperature to remove any unencapsulated DNA from the material surface. Leaching of entrapped aptamers from the materials was determined by comparing the total fluorescence intensity prior to any washing to that of washed materials, as well as the fluorescence intensity of the combined wash solutions for all three washes. Fluorescence anisotropy of the wash solutions were also measured. Following washing, materials were incubated at 25° C. for 10 min in the plate reader prior to target addition to the overlaid buffer solution and fluorescence measurements. This experiment was also repeated with the monomeric versions of each aptamer, complexed in the same 1:1:6 ratio.

To test the sensitivity of the aptamer complexes in materials, following incubation and washing, ATP was added to the ATP-binding concatemer at final concentrations of 0-3 mM, while PDGF was added to the PDGF-binding concatemer at a final concentration range of 0-300 nM (2 μL of each analyte solution was added at the appropriate concentration). The selectivity of entrapped concatemers to bind their specific target over structurally-related molecules was also assessed. The ATP-binding concatemer was incubated with ATP, CTP, GTP or UTP at a final concentration of 2 mM, while the concatemeric PDGF aptamer was incubated with PDGF, IGF-I, EGF or BSA at a final concentration of 200 nM.

To assess nuclease resistance upon entrapment, 1 U of DNase I was added directly to solution samples or the overlaying buffer of macroporous material samples containing either the ATP-binding or PDGF-binding concatemer complex. Degradation will cause liberation of FDNA and/or QDNA, resulting in a significant increase in fluorescence intensity. Degradation was thus assessed by measuring the time-dependent increases in fluorescence emission over a period of 60 min following addition of DNase I, and was compared to the emission changes for control samples without added DNase I.

Preparation of Monolithic Chromatography Columns. Both monomeric and concatemeric aptamers were entrapped in monolithic columns, where the original QDNA quencher moiety was replaced with a fluorescein (FAM)- or Cy5-labelled strand with an identical sequence to produce F′DNA or F″DNA, respectively. The aptamers were combined with F′DNA (or F″DNA) in a 1:6 molar ratio at 2× final concentration in 2× PEG-doped assay buffer, heated at 90° C. for 5 min, and cooled at room temperature to anneal the F′DNA to the aptamer.

Monolithic columns were prepared by mixing the 60% TMOS—40% MTMS (v/v) sol in a 1:1 volume ratio with the 2× PEG-doped assay buffer and immediately loading the mixture into 2 m of 250 μm i.d. fused-silica capillary. The final composition of the solution was 5% PEG (6 000 Da) containing either no DNA molecules, 600 nM F′DNA only, or 100 nM aptamer (concatemeric or monomeric aptamer) complexed with 600 nM F′DNA (or F″DNA) in 1× assay buffer. Columns were laid flat at room temperature in air for 3 h for gelation and preliminary aging to occur. Then, the ends of the capillaries were immersed in Eppendorf tubes containing 1× assay buffer and covered with Parafilm™ to prevent evaporation. The monoliths were further aged for at least 3-14 days at 4° C. Columns were then cut into 10-cm pieces (discarding 10 cm segments from each end) and attached to an Eksigent 2D nanoLC pump with autosampler (Dublin, Calif.) using standard Upchurch Scientific fittings. Assay buffer was delivered to the column at a flow rate of 1-30 μL/min and compared to empty capillaries in order to measure backpressure and column robustness. For flow-through sensor assays, columns were first conditioned using 8 bed volumes of buffer to remove any free PEG. Assay buffer was introduced to the column at a flow rate of 1 μL/min and two 20 μL fractions were collected in Eppendorf tubes. Either ATP or PDGF in buffer (at final concentrations of 0-3 mM for ATP or 0-300 nM for PDGF), human serum with or without 200 nM PDGF, or a mixed solution of 2 mM ATP and 200 nM PDGF in buffer was then added to the column using the autosampler and a third 20 μL fraction was collected. Buffer was then re-introduced into the column and a final fourth fraction of 20 μL volume was collected.

Fluorescence Intensity and Anisotropy Measurements. All fluorescence measurements were performed using a Tecan Infinite® M1000 platereader in fluorescence mode. Excitation was done at 490 nm (5-nm bandpass) and emission was measured at 520 nm (5-nm bandpass) with a 20 μs integration time using the bottom-read setting. Kinetic measurements in solution and monolithic disks were performed to assess signal response upon addition of a given target (or DNase I) using fluorescence intensity reads every 1 min for both baseline (before target/DNase addition; 10 min) and assay (after addition of target/DNase; 1 h) measurements, with orbital shaking of 2.5 mm amplitude for 5 s between each measurement to ensure proper mixing. Raw fluorescence intensity measurements were normalized to F/F₀ where F is the endpoint fluorescence intensity and F₀ is the initial fluorescence intensity prior to QDNA/target addition. Fluorescence scans of collected column fractions were performed using an excitation wavelength of 490 nm (5-nm bandpass) and measuring emission from 500-560 nm (5 nm bandpass) for fluorescein or an excitation wavelength of 645 nm (5 nm bandpass) and measuring emission from 655-715 nm (5-nm bandpass) for Cy5, using bottom-read mode. Fluorescence anisotropy measurements of monolithic disk wash solutions were performed using a 470 nm excitation wavelength and 520 nm emission wavelength (5 nm bandpass) in top-read mode and corrected for the instrumental G-factor. All assays were carried out in triplicate with background fluorescence subtraction at 25° C.

DLS Measurements. DNA sizing was performed using a Malvern Instruments Zatasizer Nano ZS to measure light scattering intensity. Samples were placed in a plastic cuvette and three separate samples of each DNA construct at 1 μM were measured using 10 runs in automatic mode at 20° C.

SEM Imaging. Samples for SEM imaging were aged in air for at least 5 days at room temperature before being cut to expose a fresh surface for mounting. Scanning electron microscopy imaging was performed using a FEI Magellan XHR 400 at 1 kV. Energy dispersive x-ray spectroscopy was performed using the same scanning electron microscope with a 5 keV beam.

### Discussion

In conclusion, the present inventors have shown that concatemeric DNA aptamers produced by RCA can be entrapped and retained within macroporous sol-gel derived materials with minimal leaching, high activity and the ability to bind high MW targets. This work also demonstrates that screening of sol-gel derived materials offers an efficient way to identify a macroporous material that can retain aptamer functionality and allow fabrication of monolithic capillary columns, which can be used as flow-based biorecognition columns. This work expands the use of sol-gel entrapped biomolecules beyond small targets toward large macromolecules such as proteins, enabling multiple new applications of sol-gel derived bio/inorganic hybrid materials.

While the present disclosure has been described with reference to what are presently considered to be the examples, it is to be understood that the disclosure is not limited to the disclosed examples. To the contrary, the disclosure is intended to cover various modifications and equivalent arrangements included within the spirit and scope of the appended claims.

All publications, patents and patent applications are herein incorporated by reference in their entirety to the same extent as if each individual publication, patent or patent application was specifically and individually indicated to be incorporated by reference in its entirety.

TABLE 1
DNA oligonucleotide sequences for aptamer reporter systems

| | | SEQ ID |
|---|---|---|
| DNA oligonucleotide | Sequence (5′→3′) | NO: |
| Linear ATP Aptamer | TGTCT TCGCC TATAG TGAAC CTTCC TCCGC AATAC | 1 |
| Circular Template | TCCCC CAGGT ATCTT TCGAC TAAGC ACC | |
| ATP Aptamer Ligation | GGCGA AGACA GGTGC TTAGT C | 2 |
| Template | | |
| ATP Aptamer Primer | GGGGG AGTAT TGCGG AGGAA | 3 |
| Linear PDGF Aptamer | TGCAG CGACT CACAG GATCA TGGTG ATGCT CTACG | 4 |
| Circular Template | TGCCG TAGCC TGCCC TTTCG ACTAC C | |
| PDGF Aptamer Ligation | GAGTC GCTGC AGGTA GTCGA A | 5 |
| Template | | |
| PDGF Aptamer Primer | CGTAG AGCAT CACCA TGATC | 6 |
| ATP aptamer FDNA | (fluorescein)CGACT AAGCA CCTGT C | 7 |
| (ATP-FDNA) | | |
| ATP aptamer QDNA (ATP- | CCCAG GTATC TT(dabcyl/fluorescein/Cy5) | 8 |
| QDNA/F′DNA/F″DNA) | | |
| ATP aptamer monomeric | TCACT ATAGG CGAAG ACAGG TGCTT AGTCG AAAGA | 9 |
| construct (ATP-Apt) | TACCT GGGGG AGTAT TGCGG AGGAA GGT | |
| PDGF aptamer FDNA | (fluorescein)GACTA CCTGC AGCGA | 10 |
| (PDGF-FDNA) | | |
| PDGF aptamer QDNA | AGCCT GCCCT TT(dabcyl/fluorescein) | 11 |
| (PDGF-QDNA/F′DNA) | | |
| PDGF aptamer monomeric | TGAGT CGCTG CAGGT AGTCG AAAGG GCAGG CTACG | 12 |
| construct (PDGF-Apt) | GCACG TAGAG CATCA CCATG ATCCT G | |

TABLE 2
The composition of the 24 macroporous sol-gel derived materials
chosen for aptamer entrapment

| Precursor | PEG MW (kDa) | [PEG] (%) |
|---|---|---|
| SS | 0.6 | 1.25 |
| SS | 0.6 | 2.5 |
| SS | 0.6 | 5 |
| SS | 1 | 1.25 |
| SS | 1 | 2.5 |
| SS | 1 | 5 |
| TMOS | 4 | 1.25 |
| TMOS | 4 | 2.5 |
| TMOS | 4 | 5 |
| TMOS | 6 | 1.25 |
| TMOS | 6 | 2.5 |
| TMOS | 6 | 5 |
| TMOS | 8 | 1.25 |
| TMOS | 8 | 2.5 |
| TMOS | 8 | 5 |
| 40% MTMS | 4 | 1.25 |
| 40% MTMS | 4 | 2.5 |
| 40% MTMS | 4 | 5 |
| 40% MTMS | 6 | 1.25 |
| 40% MTMS | 6 | 2.5 |
| 40% MTMS | 6 | 5 |
| 40% MTMS | 8 | 1.25 |
| 40% MTMS | 8 | 2.5 |
| 40% MTMS | 8 | 5 |

TABLE 3
Fluorescence polarization from ATP or PDGF concatemer,
monomer and FDNA in solution or leached from materials.

| | Solution (mP) | Leached (mP) |
|---|---|---|
| ATP Concatemer | 110 ± 2 | 70 ± 4 |
| ATP Monomer | 105 ± 1 | 107 ± 6 |
| ATP FDNA only | 61 ± 2 | 66 ± 3 |
| PDGF Concatemer | 122 ± 1 | 81 ± 9 |
| PDGF Monomer | 111 ± 1 | 116 ± 7 |
| PDGF FDNA only | 69 ± 1 | 76 ± 4 |

### REFERENCES

[1] a) D. Avnir, S. Braun, O. Lev, M. Ottolenghi, Chem. Mater. 1994, 6, 1605; b) R. B. Bhatia, C. J. Brinker, A. K. Gupta, A. K. Singh, Chem. Mater. 2000, 12, 2434; c) V. B. Kandimalla, V. S. Tripathi, H. Ju, Crit. Rev. Anal. Chem. 2006, 36, 73; d) D. Avnir, T. Coradin, O. Lev, J. Livage, J. Mater. Chem. 2006, 16, 1013; e) J. D. Brennan, Acc. Chem. Res. 2007, 40, 827; f) M. R. N. Monton, E. M. Forsberg, J. D. Brennan, Chem. Mater. 2011, 24, 796; g) E. M. Forsberg, C. Sicard, J. D. Brennan, Annu. Rev. Anal. Chem. 2014, 7, 337.
[2] J. A. Cruz-Aguado, Y. Chen, Z. Zhang, M. A. Brook, J. D. Brennan, Anal. Chem. 2004, 76, 4182.
[3] a) R. J. Hodgson, Y. Chen, Z. Zhang, D. Tleugabulova, H. Long, X. M. Zhao, M. Organ, M. A. Brook, J. D. Brennan, Anal. Chem. 2004, 76, 2780; b) N. Rupcich, R. Nutiu, Y. Li, J. D. Brennan, Anal. Chem. 2005, 77, 4300; c) T. R. Besanger, R. J. Hodgson, D. Guillon, J. D. Brennan, Anal. Chim. Acta 2006, 561, 107; d) T. R. Besanger, R. J. Hodgson, J. R. Green, J. D. Brennan, Anal. Chim. Acta 2006, 564, 106; e) Y. T. Shen, G. Mackey, N. Rupcich, D. Gloster, W. Chiuman, Y. F. Li, J. D. Brennan, Anal. Chem. 2007, 79, 3494; f) E. M. Forsberg, J. R. A. Green, J. D. Brennan, Anal. Chem. 2011, 83, 5230; g) C. Carrasquilla, P. S. Lau, Y. Li, J. D. Brennan, J. Am. Chem. Soc. 2012, 134, 10998.
[4] a) Q. Zhao, X.-F. Li, X. C. Le, Anal. Chem. 2008, 80, 3915; b) Q. Zhao, X.-F. Li, Y. Shao, X. C. Le, Anal. Chem. 2008, 80, 7586; c) N. Deng, Z. Liang, Y. Liang, Z. Sui, L. Zhang, Q. Wu, K. Yang, L. Zhang, Y. Zhang, Anal. Chem. 2012, 84, 10186; d) E. M. Forsberg, J. D. Brennan, Anal. Chem. 2014, 86, 8457; e) Z. Wang, J. C. Zhao, H. Z. Lian, H. Y. Chen, Talanta 2015, 138, 52.
[5] a) A. Fire, S. Q. Xu, Proc. Natl. Acad. Sci. U.S.A. 1995, 92, 4641; b) D. Y. Liu, S. L. Daubendiek, M. A. Zillman, K. Ryan, E. T. Kool, J. Am. Chem. Soc. 1996, 118, 1587.
[6] C. Carrasquilla, J. R. L. Little, Y. Li, J. D. Brennan, Chem. Eur. J. 2015, 21, 7369.
[7] A. M. E. Smith, J. Fortuna, E. M. Forsberg, J. D. Brennan, RSC Advances 2014, 4, 15952.
[8] C. Carrasquilla, J. D. Brennan, Methods 2013, 63, 255.
[9] a) D. E. Huizenga, J. W. Szostak, Biochemistry 1995, 34, 656; b) L. S. Green, D. Jellinek, R. Jenison, A. Ostman, C. H. Heldin, N. Janjic, Biochemistry 1996, 35, 14413.
[10] a) R. Nutiu, Y. Li, J. Am. Chem. Soc. 2003, 125, 4771; b) R. Nutiu, Y. Li, Chem. Eur. J. 2004, 10, 1868; c) R. Nutiu, Y. Li, Methods 2005, 37, 16.
[11] a) P. Del Vecchio, D. Esposito, L. Ricchi, G. Barone, Int. J. Biol. Macromol. 1999, 24, 361; b) G. Bonner, A. M. Klibanov, Biotechnol. Bioeng. 2000, 68, 339.
[12] C. Y. Hui, Y. Li, J. D. Brennan, Chem. Mater. 2014, 26, 1896.
[13] a) D. C. Schriemer, D. R. Bundle, L. Li, O. Hindsgaul, Angew. Chem. Int. Ed. 1998, 37, 3383; b) E. Peyrin, J. Sep. Sci. 2009, 32, 1531; c) V. Pichon, F. Brothier, A. Combes, Anal. Bioanal. Chem. 2015, 407, 681; d) F. Du, L. Guo, Q. Qin, X. Zheng, G. Ruan, J. Li, G. Li, TrAC, Trends Anal. Chem. 2015, 67, 134.
[14] Liu D. M.; Chen I. W. Acta Mater. 1999, 47, 4535.

1. A bio/inorganic hybrid material comprising:
a) a macroporous sol-gel; and
b) one or more concatemeric nucleic acid molecules entrapped within a) having a molecular weight greater than 10,000 Da and comprising one or more tandem repeating functional nucleic acid sequences.

2. The bio/inorganic hybrid material of claim 1, wherein the macroporous sol-gel is derived silicate, organosilicate composite or other metal oxide or mixed metal oxide composite.

3. The bio/inorganic hybrid material of claim 1, wherein the concatemeric nucleic acid molecules comprise tandem repeating functional sequences for one or more RNA aptamers or DNA aptamers or DNAzymes or aptazymes, or a combination thereof.

4. A biosensor comprising
a) the bio/inorganic hybrid material of claim 1, further comprising
b) nucleic acid molecules complementary to at least one portion of the concatemeric nucleic acid molecule labelled with a detectable label for detection of an analyte.

5. The biosensor of claim 4, wherein the detectable label for detection of the analyte is suitable for a fluorescent system, a colorimetric system, Raman, infrared or other optical system, and an electrochemical system.

6. The biosensor of claim 5, wherein the detectable label for detection of the analyte comprises a fluorescent system.

7. The biosensor of claim 6 wherein b) comprises fluorophore labeled nucleic acid molecules complementary to a portion of the concatemeric nucleic acid molecules and further comprising nucleic acid molecules complementary to a second portion of the concatemeric nucleic acid molecules conjugated to a quencher; wherein the quencher quenches the fluorophore in the absence of the analyte.

8. A method for preparing a bio/inorganic hybrid material, comprising:
a) combining concatemeric nucleic acid molecules with a sol-gel precursor; and
b) incubating a) under conditions to form a macroporous metal oxide or organically-modified metal oxide gel.

9. The method of claim 8, wherein the sol-gel precursor comprises a mixture of tetramethoxysilane and methyltrimethoxysilane in a 60:40 volume percent ratio with 5 percent weight-to-volume 6,000 Da poly(ethylene glycol).

10. The method of claim 8, wherein b) comprises forming the macroporous metal oxide or organically-modified metal oxide gel into bulk monoliths, monolithic capillary columns, thin films, or arrays.

11. A method of preparing a biosensor comprising
a) combining concatemeric nucleic acid molecules and nucleic acid molecules complementary to a portion of the concatemeric nucleic acid molecule labelled with a detectable label for detection of an analyte; and b) mixing with a sol-gel precursor under conditions to form a macroporous metal oxide or organically-modified metal oxide gel.

12. The method of claim 11, further comprising in a) combining nucleic acid molecules complementary to a second portion of the concatemeric nucleic acid molecule conjugated to a quencher; wherein the quencher quenches the fluorophore in the absence of the analyte.

13. A monolithic capillary column comprising the bio/inorganic hybrid material of claim 1, within a hollow fused silica capillary.

14. A monolithic capillary column comprising the biosensor of claim 4, within a hollow fused silica capillary.

15. A method of detecting one or more analytes in a sample, comprising:
a) mixing the sample with the biosensor material of claim 7, wherein the analyte binds to the concatemeric molecules, displacing the quencher conjugated nucleic acid molecules; and
b) detecting the fluorophore-labelled nucleic acid molecules;
wherein detecting the fluorophore-labelled nucleic acid molecules indicates the presence of the one or more analytes.

16. The method of claim 15, wherein the analyte is selected from metal ions, small molecules, drugs, hormonal growth factors, biomolecules, toxins, peptides, proteins, viruses, bacteria, and cells.

17. A method of detecting one or more analytes in a sample, comprising:
a) flowing the sample through a monolithic column comprising the biosensor material of claim 6; and
b) monitoring for detection, wherein a positive result indicates the presence of the one or more analytes in the sample.

18. The method of claim 17, wherein the analyte is selected from metal ions, small molecules, drugs, hormonal growth factors, biomolecules, toxins, peptides, proteins, viruses, bacteria, and cells.

19. The method of claim 17, wherein in a) the analyte binds to the concatemeric nucleic acid molecules displacing the labeled nucleic acid molecules complementary to a portion of the concatemeric nucleic acid molecule; and b) comprises collecting eluate from the column and detecting the fluorescence of the eluate.

20. A method of separating one or more target molecules from a sample, comprising:
a) capturing the one or more target molecules using a monolithic column comprising the bio/inorganic hybrid material of claim 1 by flowing the sample through said monolithic column, wherein the bio/inorganic material comprises concatemeric nucleic acid molecules that are able to bind to the target molecule; and
b) optionally isolating the one or more molecules from the monolithic column.

21. The method of claim 20, wherein the target molecule is selected from metal ions, small molecules, drugs, hormonal growth factors, biomolecules, toxins, peptides, proteins, viruses, bacteria, and cells.


SEQUENCE LISTING (12 sequences)

SEQ ID NO: 1 - DNA, 63 bp, Artificial Sequence
  Synthetic Construct
tgtcttcgcctatagtgaaccttcctccgcaatactcccccaggtatctttcgactaagcacc

SEQ ID NO: 2 - DNA, 21 bp, Artificial Sequence
  Synthetic Construct
ggcgaagacaggtgcttagtc

SEQ ID NO: 3 - DNA, 20 bp, Artificial Sequence
  Synthetic Construct
gggggagtattgcggaggaa

SEQ ID NO: 4 - DNA, 61 bp, Artificial Sequence
  Synthetic Construct
tgcagcgactcacaggatcatggtgatgctctacgtgccgtagcctgccctttcgactacc

SEQ ID NO: 5 - DNA, 21 bp, Artificial Sequence
  Synthetic Construct
gagtcgctgcaggtagtcgaa

SEQ ID NO: 6 - DNA, 20 bp, Artificial Sequence
  Synthetic Construct
cgtagagcatcaccatgatc

SEQ ID NO: 7 - DNA, 16 bp, Artificial Sequence
  Synthetic Construct
cgactaagcacctgtc

SEQ ID NO: 8 - DNA, 12 bp, Artificial Sequence
  Synthetic Construct
cccaggtatctt

SEQ ID NO: 9 - DNA, 63 bp, Artificial Sequence
  Synthetic Construct
tcactataggcgaagacaggtgcttagtcgaaagatacctgggggagtattgcggaggaaggt

SEQ ID NO: 10 - DNA, 15 bp, Artificial Sequence
  Synthetic Construct
gactacctgcagcga

SEQ ID NO: 11 - DNA, 12 bp, Artificial Sequence
  Synthetic Construct
agcctgcccttt

SEQ ID NO: 12 - DNA, 61 bp, Artificial Sequence
  Synthetic Construct
tgagtcgctgcaggtagtcgaaagggcaggctacggcacgtagagcatcaccatgatcctg